(19) United States
(12) Patent Application Publication
(10) Pub. No.: US 2024/0023573 A1
(43) Pub. Date: Jan. 25, 2024

(54) COLOSTRUM HYDROLYSIS PRODUCT WITH IMPROVED L-TRYPTOPHAN CONTENT, METHOD FOR MANUFACTURING THE SAME AND USE THEREOF

(71) Applicant: FARMSKIN INC., Cheongju-si (KR)

(72) Inventors: Taeil KWAK, Cheongju-si (KR); Junhyuk KIM, Seoul (KR); Kwang Il KIM, Seoul (KR); Woo Ram LEE, Sejong-si (KR); Yang Hee JO, Cheongju-si (KR); Jaehyeok CHOI, Cheongju-si (KR)

(21) Appl. No.: 18/046,617

(22) Filed: Oct. 14, 2022

(30) Foreign Application Priority Data
Jul. 25, 2022 (KR) 10-2022-0091815

(51) Int. Cl. A23J 3/34; A61Q 19/00; A23L 33/18; A61Q 19/02; A61K 8/98

(52) U.S. Cl. CPC A23J 3/341; A61Q 19/00; A23L 33/18; A61Q 19/02; A61K 8/986

(57) ABSTRACT

The present invention provides a colostrum hydrolysate having a content of L-tryptophan improved by hydrolysis of colostrum using a proteolytic enzyme, a method for production of the same, and a whitening composition including the above hydrolysate as an active ingredient. The colostrum hydrolysate according to the present invention has an advantage in that low molecular weight components such as peptide, L-tryptophan, etc. are generated by hydrolyzing protein contained in a colostrum extract using hydrolase, thereby enabling easy absorption thereof to the human body. The colostrum hydrolysate may be usefully utilized in cosmetics, foods, medicines, feed industry and the like.

### CROSS REFERENCE TO RELATED APPLICATIONS

This application claims the priority benefit of Korean Patent Application No. 10-2022-0091815 filed on Jul. 25, 2022, the entirety of which is incorporated by reference herein.

### BACKGROUND OF THE INVENTION

### 1. Field of the Invention

The present invention relates to a colostrum hydrolysis product (“colostrum hydrolysate”) with improved L-tryptophan content, a method for production of the same, and use thereof, and more particularly, to a colostrum hydrolysate having a content of L-tryptophan improved by hydrolysis of colostrum using a proteolytic enzyme, a method for production of the same, and a whitening composition containing the same as an active ingredient.

### 2. Description of the Related Art

Colostrum is the milk produced for the first few days after giving birth, which is a peptide-rich natural source containing a large amount of nutrients, immune globulin, etc., and having anti-bacterial activity and growth factors. Mostly, due to high immune globulin and casein values, the colostrum has a total protein content higher than that in normal milk.

The protein component may be divided into whey protein as a water-soluble component and casein as an insoluble protein, and both of the above components impart nutrition and physiological activity. The casein includes peptides having opioid-type activity, and functions as a competitive matrix, such that IgG, growth factors and other biologically active proteins may be protected from intestinal protein decomposition.

The whey protein contains a variety of growth factors including immune globulin, lactoferrin, lactoperoxidase, α-lactalbumin, α-lactoglobulin, etc. Among them, α-lactalbumin exists in the colostrum at a high concentration, and occupies about ¼ of the total protein content having high essential amino acid content. Further, β-lactoglobulin is composed of 162 amino acids and serves as a good source of essential amino acids.

There are total 22 amino acids, and among them, 8 amino acids are not produced by the human body itself, but may be obtained from an outside, which are called essential amino acids. Types of essential amino acids may include tryptophan, isoleucine, valine, methionine, lysine, threonine, leucine and phenylalanine.

Among the essential amino acids, tryptophan cannot be synthesized by most of animals themselves including the human being, which thereby may be obtained by food intake. Tryptophan is used as a precursor for biosynthesis of proteins, in which 5-HTP is converted into a sleeping hormone, substances such as serotonin or niacin having obesity improvement effects, etc. through metabolic materials. The component converted into the above substance is a natural relaxant to induce normal sleeping so as to relieve insomnia, and functions to treat migraine while reducing a cholesterol level, thereby being helpful for human health.

The colostrum contains various ingredients unlike normal milk and is recognized as a preferable substance, however, the colostrum collected during initial time of production are likely corruptible and entail difficulties in storage. Further, it is difficult to process the colostrum, hence causing a great limitation in utilizing the same.

Therefore, the present inventors researched techniques for processing the colostrum, established colostrum processing skills based on the above research, and have filed various patent applications including, for example, Korean Patent Laid-Open Publication No. 10-2018-0108504 and granted a patent. Specifically, the processed product has been registered as a raw material in the International Cosmetic Ingredient Dictionary and Handbook (ICID).

In order to extract diverse components from natural resources, different methods are being used in the art. Among them, a chemical extraction process using an organic solvent is utilized the most. Such a chemical process may encounter safety-related problems due to generation or remaining of harmful substances. In fact, since this is not an eco-friendly process, it is being sought for a variety of methods to overcome or compensate the above-described problems. As an alternative proposal, a bioconversion process using a biocatalyst such as enzymes has been on the rise.

Bioconversion refers to a technology that converts the existing material into a different one using functions of a biocatalyst such as microorganisms, enzymes, etc. The enzyme used in the above process is a non-toxic and eco-friendly ingredient, and may convert an insoluble component into a water-soluble component thus to induce effects of increasing extraction efficiency.

There are patents regarding application of bioconversion skills to increase the content of a specific ingredient or to use such specific ingredients as a composition for food and/or cosmetics, for example: Korean Patent Registration Publication No. 10-1822752 which proposes a method for preparation of deer antler enzyme hydrolysis extract containing high bioactive ingredients of deer antler, and the antler enzyme hydrolysis extract prepared thereby; Korean Patent Registration Publication No. 10-1705481 which proposes a method for preparation of oyster mushroom extract with improved Ergothioneine content, and the oyster mushroom extract prepared thereby; Korean Patent Registration Publication No. 10-1161634 which proposes a method for preparation of antler fermented liquor with improved contents of specific free amino acids, and the antler fermented liquor prepared thereby; and Korean Patent Registration Publication No. 10-2104225 which proposes a method for preparation of Codonopsis lanceolata callus extract, wherein contents of active ingredients are increased using hydrolase and an ultra-high pressure emulsifying machine.

However, according to the above conventional technologies, the processed product was prepared through fermentation as one of the bioconversion skills, and ingredients modified or changed through fermentation were not identified. Accordingly, it is believed that such modified or changed ingredients after preparing a new processed product using hydrolase during bioconversion would need to be identified.

### PRIOR ART DOCUMENT

### Patent Document

(Patent Document 1) Korean Patent Laid-Open Publication No. 10-2018-0108504
(Patent Document 2) Korean Patent Registration Publication No. 10-1822752
(Patent Document 3) Korean Patent Registration Publication No. 10-1704381
(Patent Document 4) Korean Patent Registration Publication No. 10-1161634
(Patent Document 5) Korean Patent Registration Publication No. 10-2104225

### SUMMARY OF THE INVENTION

The present invention has been proposed to overcome the conventional problems described above, and based on results of the researches for availability of colostrum ingredients, it is a task of the present invention to provide a colostrum hydrolysis product which is obtained by hydrolysis of colostrum and has improved L-tryptophan content, and a new use thereof.

Accordingly, an object of the present invention is to provide a colostrum hydrolysis product (“colostrum hydrolysate”) which is obtained by hydrolysis of the colostrum and has improved L-tryptophan content.

In addition, another object of the present invention is to provide a method for production of a colostrum hydrolysate with improved L-tryptophan through hydrolysis of the colostrum.

Further, another object of the present invention is to provide a use of colostrum hydrolysate with improved L-tryptophan content, which may be applied to a whitening composition.

In order to accomplish the above objects, the present invention provides a colostrum hydrolysate with a content of L-tryptophan improved by hydrolysis of colostrum.

According to an embodiment of the present invention, the hydrolysis may be performed to hydrolyze the colostrum using a protein hydrolytic enzyme (“proteolytic enzyme”).

According to an embodiment of the present invention, the proteolytic enzyme may include any one or more selected from the group consisting of flavourzyme, protamex, papain, protease, nutrease, alcalase, lipase, phosphatase, DNA glycosylase, amylase, nuclease and helicase.

According to an embodiment of the present invention, the content of the L-tryptophan may be 10 ppm or more.

According to an embodiment of the present invention, the content of the L-tryptophan may range from 10 to 400 ppm.

According to an embodiment of the present invention, the proteolytic enzyme may be alcalase, and the content of the L-tryptophan may range from 20 to 50 ppm.

In order to accomplish the above objects, the present invention provides a method for production of colostrum hydrolysate with improved L-tryptophan content, which includes: (1) adding a proteolytic enzyme to a colostrum extract to conduct hydrolysis; and (2) inactivating the proteolytic enzyme.

According to a preferred embodiment of the present invention, the proteolytic enzyme used herein may include any one or more selected from the group consisting of flavourzyme, protamex, papain, protease, nutrease, alcalase, lipase, phosphatase, DNA glycosylase, amylase, nuclease and helicase.

In order to accomplish the above objects, the present invention provides a whitening composition including the colostrum hydrolysate with improved L-tryptophan content as an active ingredient.

According to an embodiment of the present invention, the composition may be a cosmetic composition, edible cosmetics, a health functional food composition, a pharmaceutical composition or a feed composition.

The colostrum hydrolysate according to the present invention may hydrolyze specific proteins contained in the colostrum extract through a hydrolytic enzyme to generate low molecular weight components such as peptide, L-tryptophan, etc., thereby having an advantage of enabling easy absorption of the above components into the human body. Further, the colostrum hydrolysate can be usefully applied to cosmetics, foods, medicines, feed industries and the like.

### BRIEF DESCRIPTION OF THE DRAWINGS

The above and other objects, features and other advantages of the present invention will be more clearly understood from the following detailed description taken in conjunction with the accompanying drawings, in which:

FIG. 1 illustrates analysis results of high performance liquid chromatography (HPLC) in regard to colostrum extract and colostrum hydrolysate prepared in Comparative Example 1 and Example 1 of the present invention, respectively, and L-tryptophan standard products;

FIG. 2 illustrates analysis results of liquid chromatography-mass spectrometer in regard to the colostrum hydrolysate and L-tryptophan prepared in Example 1 of the present invention, wherein data on the upper side shows the result of the colostrum hydrolysate while data on the lower side exhibits the result of L-tryptophan standard product as an indicator component;

FIG. 3 illustrates analysis results of HPLC in regard to L-tryptophan of a colostrum hydrolysate prepared in Example 4 of the present invention for each type of proteolytic enzymes; and

FIG. 4 illustrates experimental results of melanin cells with respect to whitening efficacy of the colostrum hydrolysate prepared in Example 4 of the present invention for each type of proteolytic enzymes, as compared to a control group.

### DETAILED DESCRIPTION OF THE INVENTION

Hereinafter, preferred embodiments of the present invention will be described in more detail.

A first embodiment of the present invention relates to a colostrum hydrolysate with a content of L-tryptophan improved by hydrolysis of colostrum.

The colostrum hydrolysate of the present invention is a hydrolysate having more excellent utility than the colostrum extract, wherein the colostrum hydrolysate becomes to have a lower molecular weight compared to the colostrum extract, therefore, has an advantage of enabling easy absorption thereof into the human body.

The colostrum hydrolysate according to the preset invention has excellent whitening effects. Therefore, when applying the inventive colostrum hydrolysate to production of a natural cosmetic composition, or the like through hydrolysis of colostrum in order to replace and compensate the existing chemical synthesis or natural component-derived whitening compositions, a whitening composition having improved whitening effects with a new use may be provided.

The present invention is initiated from a purpose of efficiently using colostrum as a raw material for hydrolysis so as to increase utility thereof.

According to a preferred embodiment of the present invention, a colostrum hydrolysate with improved L-tryptophan content may be provided through hydrolysis of colostrum.

According to a preferred embodiment of the present invention, the colostrum hydrolysate exhibits more excellent whitening effects than hydrolysates of other dairy products such as milk, whey or fermented products thereof and the like.

According to the present invention, the hydrolysis applied to the above colostrum hydrolysate is preferably a hydrolysis performed using a hydrolytic enzyme.

According to a preferred embodiment of the present invention, as the hydrolytic enzyme applicable to hydrolysis of colostrum, the proteolytic enzyme is preferably used.

According to a preferred embodiment of the present invention, the proteolytic enzyme used herein may include any one or more selected from the group consisting of flavourzyme, protamex, papain, protease, nutrease, alcalase, lipase, phosphatase, DNA glycosylase, amylase, nuclease and helicase.

Preferably, as the proteolytic enzyme, one or more of flavourzyme, protamex, protease and alcalase, and more preferably, one or more of flavourzyme, protease and alcalase are used, but they are not limited thereto.

The protease is preferably a protease derived from Aspergillus, but it is not limited thereto.

The colostrum used in the present invention is preferably milk made typically within 3 days after delivery, but it is not limited thereto.

The colostrum used in the present invention may be colostrum of domestic animals such as cow, nanny goat, mountain goat, etc., but it is not limited thereto.

In the present invention, among various types of colostrum, cow colostrum may be used, wherein the colostrum is normally a concept including colostrum of livestock such as nanny goat, mountain goat, etc., but it is not limited thereto.

According to the present invention, colostrum free of fat and/or casein may be used as a raw material.

According to a preferred embodiment of the present invention, a method for removing fat from the colostrum may include general centrifugation, filtration, use of a fat coagulant or the like. In order to remove casein or the like, for example, an enzyme composite serving as a coagulase may be used. Specifically, for example, rennet is preferably used.

According to a preferred embodiment of the present invention, the content of the L-tryptophan may range from 10 to 400 ppm. According to a preferred embodiment of the present invention, the proteolytic enzyme may be alcalase, and the content of the L-tryptophan may range from 20 to 50 ppm, but it is not limited thereto.

A second embodiment of the present invention of the present invention relates to a method for production of colostrum hydrolysate with improved L-tryptophan content, which includes: (1) adding a proteolytic enzyme to a colostrum extract to conduct hydrolysis; and (2) inactivating the proteolytic enzyme.

According to a preferred embodiment of the present invention, the proteolytic enzyme used herein may include any one or more selected from the group consisting of flavourzyme, protamex, papain, protease, nutrease, alcalase, lipase, phosphatase, DNA glycosylase, amylase, nuclease and helicase.

Preferably, as the proteolytic enzyme, one or more of flavourzyme, protamex, protease and alcalase, and more preferably, one or more of flavourzyme, protease and alcalase are used, but they are not limited thereto.

The protease is preferably Aspergillus-derived protease, but it is not limited thereto.

According to a preferred embodiment of the present invention, the hydrolysis may be performed at 40 to 65° C., preferably 50 to 60° C., more preferably 53 to 57° C. for to 5 hours, preferably 1 to 4 hours, and more preferably 2 to 4 hours. If the hydrolysis temperature is beyond the above range, hydrolysis may not be efficiently performed, hence causing a problem of affecting adversely on the desired improvement of L-tryptophan.

According to an embodiment of the present invention, the inactivation described above may be performed at 70 to 95° C., preferably 80 to 90° C., more preferably 83 to 87° C., and most preferably 85° C. If the inactivation temperature is beyond the above range, it may entail a problem of affecting adversely on the desired improvement of L-tryptophan.

The hydrolysis process of the present invention may be performed by preparing colostrum with a concentration of 1 to 90% by weight (“wt. %”) as a base material and then adding 0.1 to 5 wt. % of a hydrolytic enzyme thereto. The obtained hydrolysate may be used after filtration.

The hydrolysate obtained through the above processes according to the present invention was confirmed to exhibit very useful properties, which are different from those of any ingredient derived from the existing colostrum- or milk-processed products. Specifically, it was confirmed to have surprisingly excellent skin whitening effects.

According to a preferred embodiment of the present invention, the hydrolysate of the present invention can exhibit remarkably excellent whitening effects even with a very small amount thereof. For example, when using the colostrum, excellent whitening effects are expressed with 0.1 to 500 μg/ml, 0.5 to 300 μg/ml, 1 to 200 μg/ml, 1 to 100 μg/ml, or even with further smaller amount such as 1 to 50 μg/ml of the colostrum.

According to a preferred embodiment of the present invention, the method may further include any one or more processes of casein removal or sterilization before or after hydrolysis.

According to a preferred embodiment of the present invention, the colostrum may be used, for example, through a step of performing sterilization at low temperature to prepare a base material. During this process, fat-free colostrum may be used as the base material. The method for removing fat from the colostrum may include general centrifugation, filtration, use of a fat coagulant or the like.

The sterilization at low temperature in the present invention may be performed according to a typical low temperature pasteurization method.

According to a preferred embodiment of the present invention, the colostrum hydrolysate of the present invention may be one in which casein is removed in any random step of the manufacturing process.

According to a preferred embodiment of the present invention, in order to remove casein, for example, an enzyme composite serving as a coagulase may be used. Specifically, for example, rennet is preferably used.

According to a preferred embodiment of the present invention, the colostrum hydrolysate may be produced after one or more further process such as filtration, sterilization, extraction or lyophilization.

The hydrolysate obtained according to the present invention may be used after removing bacteria possibly present therein through filtration twice or several times after production thereof.

A third embodiment of the present invention relates to a whitening composition that includes the colostrum hydrolysate with improved L-tryptophan content as an active ingredient.

According to a preferred embodiment of the present invention, the composition may include a cosmetic composition, edible cosmetics, a health functional food composition, a pharmaceutical composition, a feed composition, and the like, but it is not limited thereto.

That is, the colostrum hydrolysate according to the present invention may exhibit skin whitening effects under quite superior conditions than the colostrum. As such, the hydrolysate of the present invention, specifically, the colostrum hydrolysate was confirmed to remarkably express, for example, effects of inhibiting melanin generation.

Melanin is a generic term for brown or black polymeric pigments derived from oxidation of phenols by oxidase, and serves to absorb light in living organisms. The colostrum hydrolysate of the present invention has remarkable effects of inhibiting synthesis of melanin cells.

Further, the hydrolysate of the present invention may exclude a disadvantage caused by casein, etc. when performing purification, and cause an increase in a content of functional ingredients, thereby exhibiting further excellent skin whitening effects, which were unpredictable, as compared to other dairy products or fermented products thereof.

According to a preferred embodiment of the present invention, specifically, the colostrum hydrolysate of the present invention may exhibit substantially and superior skin whitening activity with respect to inhibition of melanin generation, even using a small amount of the hydrolysate unlike the existing whitening compositions. At the same time, various ingredients contained in the hydrolysate of the present invention may improve skin environments so that natural cosmetics can produce clean skin environments and prevent pigmentation from occurring on the skin, and thereby being very desirably used as cosmetics for skin whitening.

Therefore, the skin whitening composition using the hydrolysate according to the present invention may be used in cosmetics, which is a novel concept of natural whitening cosmetics never known in the conventional art.

According to the present invention, such a whitening composition using the hydrolysate described above may inhibit generation of melanin so as to exhibit very significant skin whitening effects.

The colostrum hydrolysate with improved L-tryptophan content of the present invention has excellent skin barrier reinforcement, immune enhancement and wrinkle improvement efficacy. Therefore, in order to replace and compensate the existing chemical synthesis or natural component-derived other compositions for skin improvement, when the above colostrum hydrolysate is applied to production of a natural cosmetic composition through hydrolysis of colostrum, a composition for skin improvement having increased skin improvement effects for new use thereof may be provided.

According to a preferred embodiment of the present invention, the colostrum hydrolysate with improved L-tryptophan content exhibits more excellent skin improvement effects, as compared to hydrolysates of other dairy products such as milk, whey or fermented products thereof and the like.

Using the colostrum hydrolysate with improved L-tryptophan content according to the present invention inhibited β-hexosaminidase, therefore, skin barrier reinforcing effects were confirmed. Further, nitrogen monoxide (NO) participating in an immune reaction was confirmed thus to demonstrate immune enhancement effects. Further, a change in activities of collagenase and elastase involved in skin wrinkle improvement was measured thus to demonstrate wrinkle improvement effects.

According to a preferred embodiment of the present invention, the colostrum hydrolysate with improved L-tryptophan content may be added to a composition for skin whitening or skin improvement in terms of various properties. For example, the above colostrum hydrolysate may be added in the form of powder, liquid, nano-capsules, composite, etc, and as a preferable example, may be added in a liquid phase.

According to a preferred embodiment of the present invention, the cosmetic composition may be one or more formulation selected from the group consisting of toilet water, nourishing lotion, nourishing cream, moisture cream, massage cream, essence, paste, mask pack, patch, gel, cream, lotion, powder, soap, cleanser, oil, foundation, make-up base, wax and spray, but it is not limited thereto.

The cosmetic composition may be provided in the form of, for example, solution, gel, solid or paste anhydrous product, emersion obtained by dispersing an oil phase in a water phase (“oil-in-water emulsion”), suspension, micro-emulsion, microcapsule, micro-granules, ionic (liposome) or non-ionic follicle dispersant, cream, skin, lotion, oil, powder, ointment, spray or concealing stick, but it is not limited thereto.

Further, in addition to the colostrum hydrolysate of the present invention, the cosmetic composition may additionally include adjuvants generally used in the field of cosmetics, for example, fatty substances, organic solvents, dis solvents, concentrating agents and gelling agents, softeners, anti-oxidative agents, suspending agents, stabilizers, foaming agents, aromatic agents, surfactants, water, ionic or non-ionic emulsifiers, fillers, metal ion inhibitors and chelating agents, preservatives, vitamins, blocking agents, wetting agents, essential oils, dyes, pigments, hydrophilic or lipophilic active agents, lipid follicles, or any other components typically used in the cosmetic field. These adjuvants may be included in an amount typically used in the cosmetic field.

With regard to the cosmetic composition of the present invention, the colostrum hydrolysate with improved L-tryptophan content according to the present invention may be added in an amount of 0.1 to 50 wt. %, and preferably 1 to 20 wt. % to a cosmetic composition generally included therein, but it is not limited thereto.

The cosmetic composition of the present invention may be used alone or in combination, or may be used in combination with other cosmetic compositions except for the inventive composition. Further, the cosmetic composition of the present invention may be used according to any conventional using method, and the number of uses thereof may vary depending on skin conditions or preference of users.

According to a preferred embodiment of the present invention, the health functional food composition may be implemented in the form of directly adding the colostrum hydrolysate with improved L-tryptophan content to various foods or using the same along with other food or food components. That is, the colostrum hydrolysate may be used in the form of a health functional food composition, and may be used according to any conventional method.

Examples of the food to which the colostrum hydrolysate described above is added may include, for example, meat, sausage, bread, chocolate, candies, snacks, confectionary, pizza, ramen, other noodles, gums, dairy products including ice creams, various soups, beverages, teas, drinks, alcoholic drinks and vitamin complex, etc. Specifically, such a health functional food may be manufactured by adding the extract of the present invention as a major component to squeezed juice, tea, jelly and juice.

In the present invention, the “health functional food” refers to a food prepared and processed in the form of tablets, capsules, powder, granules, liquid, pills, etc. using raw materials or components having functionality useful for the human body, and may include edible cosmetics, inner beauty compositions and the like. As used herein, the term “functionality” refers to regulating nutrients or imparting effects useful for health care such as physiological action or bio-activity with respect to the structure and function of the human body.

The health functional food of the present invention can be manufactured by any conventional method commonly used in the art, and may be produced by adding raw materials and components generally added in the art during manufacturing. Further, formulations of the health functional food may be manufactured without limitation thereof so far as they are recognized as health functional foods.

The health functional food composition of the present invention may be manufactured in a variety of formulations, and has an advantage in that, unlike common medicines, foods are used as the raw material thus to eliminate possibility of adverse effects occurring when a medicine is taken for a long time. Further, the health functional food of the present invention is excellent in carrying, and may be possibly taken as an adjuvant to improve effects of an antioxidant.

The health functional food composition of the present invention may further include a sitologically acceptable food supplement additive in addition to the active ingredient, and a mixing amount of the active ingredient may be suitably determined according to use purposes (prevention, health or therapeutic treatment).

Further, according to a preferred embodiment of the present invention, the pharmaceutical composition may be manufactured using a pharmaceutically suitable and physiologically acceptable adjuvant in addition to the active ingredient. In this case, the adjuvant used herein may include excipients, disintegrants, sweetening agents, binders, coatings, swelling agents, lubricants, glidants or flavors.

The formulations of the pharmaceutical composition may include granules, powder, tablets, coated tablets, capsules, suppository, liquid, syrup, squeeze juice, suspension, emulsion, medicinal drops, injectable solution or the like. For example, for a pharmaceutical preparation in the tablet or capsule form, the active ingredient may be combined with an oral, non-toxic and pharmaceutically acceptable inactivated carrier such as ethanol, glycerol, water, etc. Further, if desired or needed, any suitable binder, lubricant, disintegrant and/or color coupler may also be included in the form of a mixture. The proper binder may include, without limitation thereof: natural sugar such as starch, gelatin, glucose or beta-lactose; natural or synthetic gums such as corn sweetener, acacia, tragacanth or sodium oleate; and sodium stearate, magnesium stearate, sodium benzoate, sodium acetate, sodium chloride and the like. The disintegrant may include, without limitation thereof, starch, methyl cellulose, agar, bentonite, xanthan gum, etc.

For the composition prepared in the form of a liquid solution, pharmaceutically acceptable carriers are suitable for sterilization and on a living body, and may include saline, sterilized water, Ringer's solution, buffer saline, albumin injectable solution, dextrose solution, maltodextrin solution, glycerol, ethanol and a mixture of one or more thereof. If necessary, other common additives such as antioxidants, buffers, bacteriostatic agents, etc. may be further added. In addition, diluents, dispersants, surfactants, binders and lubricants may be additionally used to produce injectable preparations such as water-soluble solution, suspension, emulsion, etc., as well as pills, capsules, granules or tablets.

The pharmaceutical composition may be administered orally or parenterally. For parenteral administration, intravenous injection, subcutaneous injection, intramuscular injection, intraperitoneal injection, transdermal administration, etc. may be used, and preferably, oral administration is selected.

The dosage of the pharmaceutical composition may vary according to factors including, for example, formulation methods, administration manners, age, body weight and sex of patients, pathological conditions, food, administration time, administration route, excretion rate and reaction susceptibility, or the like, and the dosage effective for desired treatment or prevention may be easily determined and prescribed by commonly skilled specialists in consideration of the above factors.

The pharmaceutical composition may be manufactured into preparations using pharmaceutically acceptable carriers and/or excipients, and the preparations may be produced in the form of unit dose or by introducing the same into a multi-dose container. At this time, the preparation may adopt the formulation of a solution in an oil or water medium, suspension or emulsion, extract, powder, granules, tablets or capsules. Further, a dispersant or stabilizer may also be added.

The colostrum hydrolysate with improved L-tryptophan content according to the present invention may also be applicable to companion animals similar to the human body. Therefore, the above colostrum hydrolysate is preferably applied to a health functional food composition for pets, an animal feed composition, an animal feed additive, and the like, so as to attain the same effects as described above, but it is not limited thereto.

Hereinafter, the present invention will be described in detail by way of examples, but the present invention is not limited to these examples.

### EXAMPLE

### <Comparative Example 1> Preparation of Colostrum Extract

After reacting colostrum in a shaking incubator at 55° C. and at a rate of 120 rpm for 2 hours, the reacted product was filtered through a 0.22 μm filter to prepare a colostrum extract.

### <Example 1> Preparation of Colostrum Hydrolysate

After adding 2% (w/w) of alcalase as a hydrolytic enzyme (“hydrolase”) to colostrum, the mixture was subjected to hydrolysis in a shaking incubator at 55° C. and at a rate of 120 rpm for 2 hours, followed by heating at 85° C. for 2 minutes and filtering the same through a 0.22 μm filter to prepare colostrum hydrolysate.

### <Example 2> Preparation of Colostrum Hydrolysate for Each Inactivation Temperature

After adding 2% (w/w) of alcalase as a hydrolase to colostrum, the mixture was subjected to hydrolysis in a shaking incubator at 55° C. and at a rate of 120 rpm for 2 hours, followed by heating at 70, 85 and 95° C. for 2 minutes, respectively, and filtering the same through a 0.22 μm filter to prepare colostrum hydrolysates.

### <Example 3> Preparation of Colostrum Hydrolysate for Each Enzyme Reaction Time

After adding 2% (w/w) of alcalase as a hydrolase to colostrum, the mixture was subjected to hydrolysis in a shaking incubator at 55° C. and at a rate of 120 rpm for 1, 2 and 4 hours, respectively, followed by heating at 85° C. for 2 minutes and filtering the same through a 0.22 μm filter to prepare colostrum hydrolysates.

### <Example 4> Preparation of Colostrum Hydrolysate for Each Type of Proteolytic Enzymes

After adding 2% (w/w) of each type of hydrolases to colostrum, the mixture was subjected to hydrolysis in a shaking incubator at 55° C. and at a rate of 120 rpm for 2 hours, followed by heating at 85° C. for 2 minutes and filtering the same through a 0.22 μm filter to prepare colostrum hydrolysate.

At this time, the hydrolases used herein were protamex, flavourzyme, alcalase and Aspergillus-derived protease.

### <Comparative Example 2> Preparation of Colostrum Hydrolysate for Each Type of Proteolytic Enzymes

Colostrum hydrolysate was prepared by the same procedure as in Example 4 above except that Bacillus-derived protease was used as the hydrolase.

### Experimental Example

In the experimental examples as described below, the following analysis methods were used.

1. Chromatogram Assay of Prepared Product Using High Performance Liquid Chromatography (HPLC)

As analysis instruments for the present experiment, high performance liquid chromatography (HPLC) (Waters, Alliance), UV/Vis detector and Xbridge C18 (250 mm×4.6 mm, 5 μm) or equivalents thereof were prepared. As supplies, HPLC vials, disposable syringes, membrane filters for filtration (PTFE, 0.45 μm) and analysis vials were prepared. As solvents for a mobile phase, acetonitrile for HPLC, methanol, distilled water and formic acid were used.

As a result of comparing the colostrum extract and the colostrum hydrolysate, a difference in chromatograms was confirmed. Further, it was confirmed that there is a component having improved content in a specific retention time of the colostrum hydrolysate.

2. Analysis of Component Having Improved Content in Colostrum Hydrolysate Using Liquid Chromatography-Mass Spectrometer

As analysis instruments for the present experiment, liquid chromatography-mass spectrometer (Agilent, 1290 Infinity & Agilent, G6550A), and EclipsePlus C18 (50 mm×2.1 mm, 1.8 μm) or equivalents thereof were prepared. As supplies, HPLC vials, disposable syringes, membrane filters for filtration (PTFE, 0.45 μm) and analysis vials were prepared. As solvents for a mobile phase, acetonitrile for HPLC, methanol, distilled water and formic acid were used.

As a result of comparing the colostrum hydrolysate and a standard product, it was confirmed that the retention time, the molecular weight, and fragmentation pattern of ions are all the same between the above two products. Further, it could be seen that the component with improved content in the colostrum hydrolysate is L-tryptophan.

3. Preparation of Standard Solution

L-tryptophan, which is an indicator component, was accurately weighed by 1 mg in 10 ml volume flask, followed by adding and dissolving the same in 1 ml of 100% distilled water to prepare a standard solution. The standard solution was desirably diluted for each concentration and used to draw a calibration curve.

### <Experimental Example 1> HPLC Comparison and Analysis of Colostrum Extract and Colostrum Hydrolysate

With regard to the colostrum extract and the colostrum hydrolysate prepared by the methods in Comparative Example 1 and Example 1, respectively, chromatograms were compared and analyzed using HPLC.

Specifically, in order to investigate changes in components of the samples in Comparative Example 1 and Example 1, an experiment was conducted according to the above analysis method 1 and results of content calculation were substituted in the calibration curve drawn in the above analysis method 3, thereby obtaining numerical values.

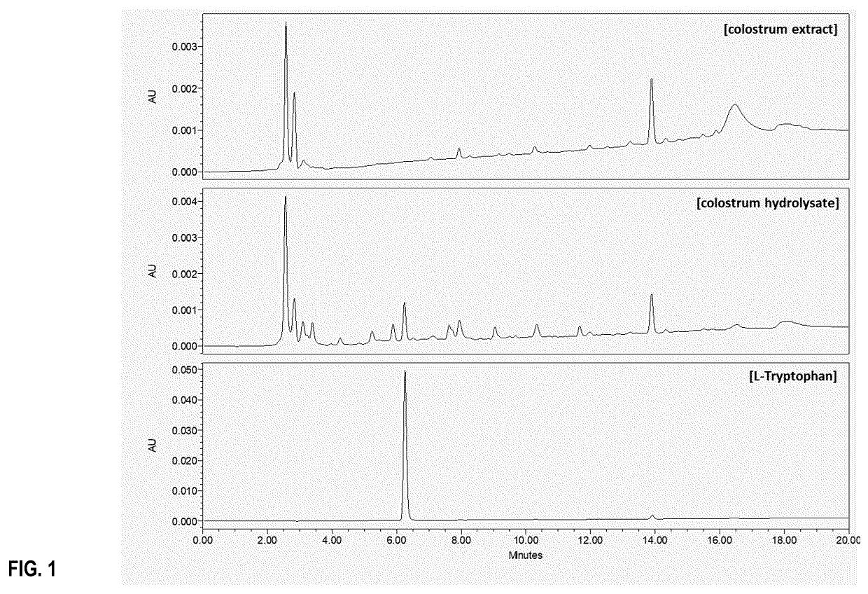

Consequently, as shown in Table 1 below and FIG. 1, it was confirmed that the content of a specific component in the sample of Example 1 is increased.

TABLE 1

| Division | Content (%) |
|---|---|
| Comparative Example 1 | N.D |
| Example 1 | 26.8 |

### <Experimental Example 2> Liquid Chromatography-Mass Spectrometer Analysis of Specific Component Having Improved Content

With regard to the colostrum hydrolysate prepared by the method in Example 1, component analysis was performed for substances having improved content by the liquid chromatography-mass spectrometer.

Specifically, in order to investigate substances having improved content contained in the sample of Example 1, an experiment was conducted by the above analysis method 2.

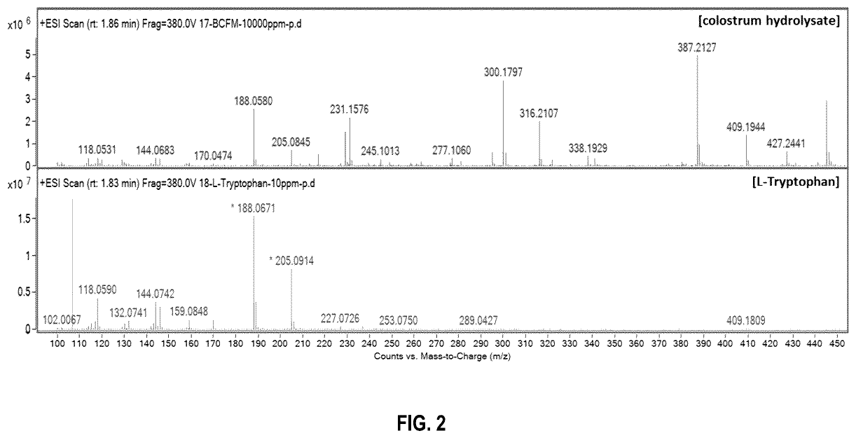

Consequently, as shown in FIG. 2, it was confirmed that the component having improved content in the colostrum hydrolysate is L-tryptophan.

### <Experimental Example 3> Comparison of L-Tryptophan Content in Colostrum Hydrolysate for Each Inactivation Temperature

In order to compare L-tryptophan content in the sample prepared by the method of Example 2, the content was measured using HPLC.

Specifically, in order to measure the content of the L-tryptophan as an indicator component in the colostrum hydrolysate for each inactivation temperature in Example 2, an experiment was conducted by the above analysis method 1. Further, results of content calculation were substituted in the calibration curve drawn in the analysis method 3, thereby obtaining numerical values.

As a result, it was determined that the optimum inactivation temperature, at which the L-tryptophan content is increased, ranged from 80 to 90° C.

### <Experimental Example 4> Comparison of L-Tryptophan Content in Colostrum Hydrolysate for Each Enzyme Reaction Time

In order to compare L-tryptophan content in the sample prepared by the method of Example 3, the content was measured using HPLC.

Specifically, in order to measure L-tryptophan content which is an indicator component of the colostrum hydrolysate for each enzyme reaction time in Example 3, an experiment was conducted by the above analysis method 1. Further, results of content calculation were substituted in the calibration curve drawn in the above analysis method 3, thereby obtaining numerical values.

As a result, it was confirmed that the optimum enzyme reaction time to increase L-tryptophan content in the sample of Example 3 ranges from 1.5 to 3 hours.

### <Experimental Example 5> Comparison of L-Tryptophan Content of Colostrum Hydrolysate for Each Type of Proteolytic Enzyme

In order to compare L-tryptophan content in the samples prepared in Example 4 and Comparative Example 2, respectively, the content was measured using HPLC. That is, in order to measure L-tryptophan, which is an indicator component of the colostrum hydrolysate for each type of the proteolytic enzymes in Example 4 and Comparative Example 2, an experiment was conducted by the above analysis method 1. Further, results of content calculation were substituted in the calibration curve drawn in the above analysis method 3, thereby obtaining numerical values.

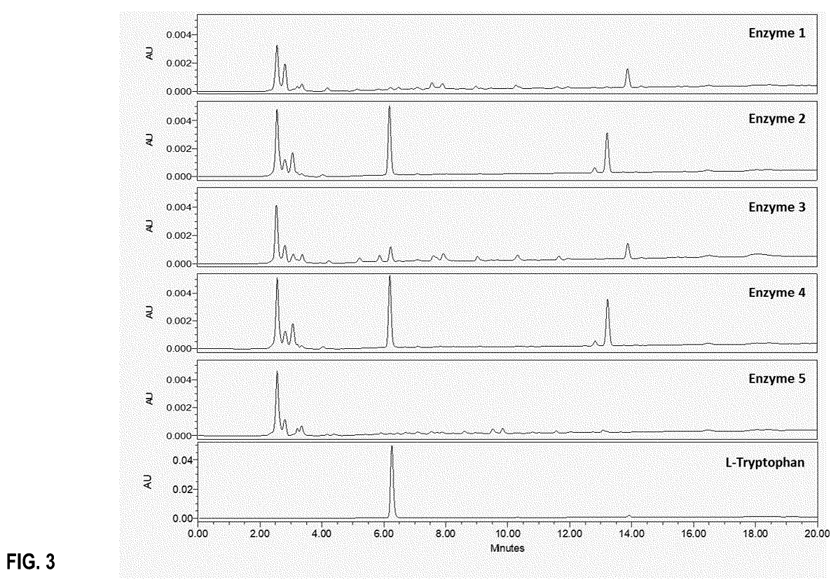

Consequently, as shown in FIG. 3 and Table 2 below, it was confirmed that, among the proteolytic enzymes to increase L-tryptophan content in the samples of Example 4 and Comparative Example 2, alcalase, flavourzyme and Aspergillus-derived protease are suitable the most.

TABLE 2

| | | L-tryptophan content |
|---|---|---|
| No. of enzyme | Proteolytic enzyme | (ppm) |
| 1 | Protamex | 16.1 |
| 2 | flavourzyme | 308.5 |
| 3 | Alcalase | 26.8 |
| 4 | Aspergillus-derived protease | 298.2 |
| 5 | Bacillus-derived protease | 7.7 |

### <Experimental Example 6> Comparison of Whitening Efficacy of Colostrum Hydrolysate for Each Type of Proteolytic Enzyme

In order to compare whitening efficacy of the samples prepared in Example 4 and Comparative Example 2, melanin inhibitory efficacy was assessed. That is, in order to compare whitening efficacy of the colostrum hydrolysate for each type of the proteolytic enzymes in Example 4 and Comparative Example 2, respectively, whitening efficacy was assessed by the following method.

Specifically, an experiment was conducted using B16F1 melanoma cells (Murine melanoma cell line). The cell line was cultured into stabilized B16F1 melanoma cells under conditions in which 5% CO₂ and a temperature of 37° C. are maintained.

The B16F1 melanoma cells cultured in a plate were isolated using 0.05% trypsin-EDTA (GIBCO BRL), dispensed on 24-well plate at a density of 2×10⁴ cells/well, followed by culturing the same for 24 hours. After replacing the cultured cell line with New-10% FBS/DMEM medium, the samples diluted for each concentration (non-treated group, control group, Example 4 and Comparative Example 2) were pre-treated on the replaced medium for 1 hour, respectively.

After the pre-treatment, the sample was treated with 100 nM alpha-melanin forming cell stimulating hormone (α-Melanocyte stimulating hormone, α-MSH). After the treatment, the treated product was cultured for 72 hours under conditions in which 5% CO₂ and a temperature of 37° C. are maintained. After culturing, the product was treated with 0.05% trypsin-EDTA to recover cells, followed by centrifugation at 12,000 rpm for 5 minutes and removing the obtained supernatant.

To the remaining residue, 100 μl of 1N sodium hydroxide containing 10% dimethyl sulfoxide (DMSO) was added, followed by leaving the mixture at 60° C. for 30 minutes, stirring and mixing again. Thereafter, melanin was quantified by measuring an absorbance at 405 nm wavelength.

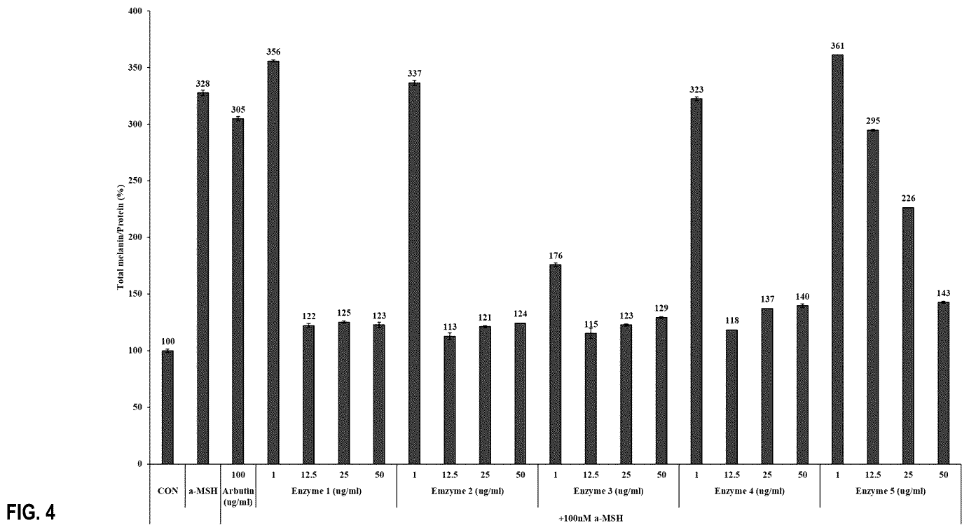

The measured O.D.405 was converted into percentage. Then, based on 100% of an amount of the melanin generated by the melanoma cells in the non-treated group, a degree of inhibition of the melanin generation by the treated sample was compared and results thereof are shown in FIG. 4. In FIG. 4, “CON” means the non-treated group, while “α-MSH” is a negative control group and “Arbutin” is a positive control group.

As a result, Example 4 exhibited superiorly excellent inhibitory effects, as compared to other control groups including the non-treated group. Specifically, in the case of the sample prepared with alcalase, it could be seen that excellent melanin inhibitory efficacy is demonstrated even at a very small amount of 1 μg/ml.

### <Experimental Example 7> Cytotoxicity Experiment

MTT assay is a measurement method of cell survival rate using dehydrogenase contained in mitochondria of a living cell. This method was used to determine whether the colostrum hydrolysate in each of the present examples has cytotoxicity or not.

The cell line used herein was B16F1 cells and, as the animal cell culture medium, Dulbecco's Modified Eagle Medium (DMEM, Gibco) including 10% FBS and 100 UL S/P was used. The cells were placed on each well of 96-well plate at 4×10³ cell/well, followed by culturing the same in an incubator at 37° C. and 5% CO₂ for 24 hours.

Thereafter, the colostrum hydrolysate was added to the cultured medium plate and further cultured for 72 hours. 0.5 mg/ml MTT solution was added to every well and reacted for 4 hours. Then, after removal of the supernatant, 100 μl of DMSO was added to dissolve formazan crystals completely. An absorbance at 520 nm was measured by a plate reader to compare cell survival rates.

The measured results were compared and shown in Table 3 below.

TABLE 3

| Division | Relative survival rate (%) |
|---|---|
| Control group | 100% |
| Experimental group | 101.3% |

As shown in Table 3 above, it could be seen that the colostrum hydrolysate according to the present invention has no cytotoxicity.

### <Experimental Example 8> Skin Stimulation Test

In order to assess a degree of skin stimulation, a human patch test was performed. Specifically, the human patch test was performed for 30 normal persons aged 20 or more. After preparing 0.001%, 0.01%, 0.1%, 1% and 10% water-soluble solutions containing the sample (Example 1), a patch was attached to the inner portion of an upper arm of each person for 48 hours using Finn chamber and, after removing the same, the above arm portion was firstly observed. Then, after 72 hours, the same arm portion was subjected to second observation so as to determine the result of skin reaction.

As a result of assessment, it was determined that the colostrum hydrolysate does not occur skin stimulation.

From the above all experimental examples, it was confirmed that hydrolysate obtained using alcalase as the proteolytic enzyme has lower L-tryptophan content as compared to other proteolytic enzymes, however, could be utilized as an indicator component of colostrum hydrolysate.

### <Preparative Example> Production of Cosmetics (Mask-Pack Composition)

With the compositions and contents listed in Table 4 below, an aqueous phase and an oil phase were dispersed and dissolved, respectively, and mixed together. Then, the mixture was subjected to saponification and cooling at room temperature, thereby producing a mask-pack composition. Further, using the mask-pack composition, a mask pack was manufactured.

TABLE 4

| Component | Content (wt. %) |
|---|---|
| Stearic acid | 6.5 |
| Myristic acid | 28.0 |
| Self-emulsion type glycerol monostearate | 3.0 |
| Colostrum hydrolysate (Example 1) | 12.0 |
| Propylene glycol | 5.0 |
| Concentrated glycerin | 10.0 |
| Sodium hydroxide | 7.0 |
| Sodium ethylenediamine tetraacetate | 0.1 |
| Pulsatilla Koreana Extract | 0.5. |
| Perfume | Trace |
| Purified water | Balance |

As such, specific portions of the subject matters in the present invention have been described in detail. Therefore, it will be obvious to those skilled in the art that the above concrete description is proposed only as preferred embodiments, however, the scope of the present invention is not limited thereto.

Accordingly, the substantial scope of the present invention will be defined by the appended claims and equivalents thereof. Simple modifications ad alterations of the present invention would be easily used by those skilled in the art, and it may be understood that such modifications or alterations are all included in the scope of the present invention.

What is claimed is:

1. A colostrum hydrolysate with a content of L-tryptophan improved by hydrolysis of colostrum.

2. The colostrum hydrolysate according to claim 1, wherein the hydrolysis is performed to hydrolyze the colostrum using a protein hydrolytic enzyme (“proteolytic enzyme”).

3. The colostrum hydrolysate according to claim 2, wherein the proteolytic enzyme includes any one or more selected from the group consisting of flavourzyme, protamex, papain, protease, nutrease, alcalase, lipase, phosphatase, DNA glycosylase, amylase, nuclease and helicase.

4. The colostrum hydrolysate according to claim 1, wherein the content of the L-tryptophan is 10 ppm or more.

5. The colostrum hydrolysate according to claim 4, wherein the content of the L-tryptophan ranges from 10 to 400 ppm.

6. The colostrum hydrolysate according to claim 1, wherein the proteolytic enzyme is alcalase, and the content of the L-tryptophan ranges from 20 to 50 ppm.

7. A method for production of colostrum hydrolysate with improved L-tryptophan content, the method comprising:
(1) adding a proteolytic enzyme to a colostrum extract to conduct hydrolysis; and
(2) inactivating the proteolytic enzyme.

8. The method according to claim 7, wherein the proteolytic enzyme includes any one or more selected from the group consisting of flavourzyme, protamex, papain, protease, nutrease, alcalase, lipase, phosphatase, DNA glycosylase, amylase, nuclease and helicase.

9. A whitening composition comprising the colostrum hydrolysate with improved L-tryptophan content according to claim 1 as an active ingredient.

10. A whitening composition comprising the colostrum hydrolysate with improved L-tryptophan content according to claim 2 as an active ingredient.

11. A whitening composition comprising the colostrum hydrolysate with improved L-tryptophan content according to claim 3 as an active ingredient.

12. A whitening composition comprising the colostrum hydrolysate with improved L-tryptophan content according to claim 4 as an active ingredient.

13. A whitening composition comprising the colostrum hydrolysate with improved L-tryptophan content according to claim 5 as an active ingredient.

14. A whitening composition comprising the colostrum hydrolysate with improved L-tryptophan content according to claim 6 as an active ingredient.

15. The whitening composition according to claim 9, wherein the composition is a cosmetic composition, edible cosmetics, a health functional food composition, a pharmaceutical composition or a feed composition.

16. The whitening composition according to claim 10, wherein the composition is a cosmetic composition, edible cosmetics, a health functional food composition, a pharmaceutical composition or a feed composition.

17. The whitening composition according to claim 11, wherein the composition is a cosmetic composition, edible cosmetics, a health functional food composition, a pharmaceutical composition or a feed composition.

18. The whitening composition according to claim 12, wherein the composition is a cosmetic composition, edible cosmetics, a health functional food composition, a pharmaceutical composition or a feed composition.

19. The whitening composition according to claim 13, wherein the composition is a cosmetic composition, edible cosmetics, a health functional food composition, a pharmaceutical composition or a feed composition.

20. The whitening composition according to claim 14, wherein the composition is a cosmetic composition, edible cosmetics, a health functional food composition, a pharmaceutical composition or a feed composition.
